(19) United States
(12) Patent Application Publication
(10) Pub. No.: US 2026/0345517 A1
(43) Pub. Date: Nov. 5, 2026

(54) DOSAGE REGIMENS FOR TREATING STARGARDT DISEASE

(71) Applicant: Alkeus Pharmaceuticals, Inc., Cambridge, MA (US)

(72) Inventor: Leonide C. Saad, Cambridge, MA (US)

(21) Appl. No.: 19/656,441

(22) Filed: Apr. 23, 2026

Related U.S. Application Data
(63) Continuation of application No. PCT/US2024/062409, filed on Dec. 31, 2024.
(60) Provisional application No. 63/617,012, filed on Jan. 2, 2024.

(51) Int. Cl. A61K 31/07; A61P 27/02

(52) U.S. Cl. CPC A61K 31/07; A61P 27/02

(57) ABSTRACT

Disclosed is a method of treating a patient with Stargardt Disease. Also disclosed is a method of delaying the progression of vision loss in a patient with Stargardt Disease. The methods comprise administering to the patient from 10 mg to 35 mg per day of gildeuretinol acetate.

### CROSS-REFERENCE TO RELATED APPLICATIONS

This application is a continuation of and claims benefit under 35 U.S.C. § 120 from PCT application PCT/US2024/062409, filed on Dec. 31, 2024, which claims the benefit of priority of U.S. Provisional Application No. 63/617,012, filed on Jan. 2, 2024, the content of which is incorporated by reference in its entirety herein.

### FEDERALLY SPONSORED RESEARCH OR DEVELOPMENT

This invention was made with government support under Grants No. R01 FD004098 and R01 FD006316 awarded by the Food and Drug Administration. The government has certain rights in the invention.

### BACKGROUND OF THE INVENTION

Stargardt Disease is a progressive condition that results in blindness and physical, psychological, and social challenges for nearly all those affected. Stargardt disease is a genetic cause of blindness in children and young adults. Stargardt disease typically presents within the first two decades of life, even though symptoms can appear during adulthood and as late as the seventh decade. The disease is usually characterized by a progressive loss of central vision causing blurry vision and, occasionally, an increasing difficulty in adapting to the dark. Most affected individuals also have impaired color vision. There is no FDA approved treatment for Stargardt disease. Symptoms of vision loss due to Stargardt disease continue to worsen over time and cannot be corrected by glasses or contact lenses because the light sensitive part of the eye, the retina, is damaged by the disease.

Gildeuretinol acetate has been proposed as a treatment for Stargardt disease (see U.S. RE 47,045). Gildeuretinol acetate is retinol acetate in which the hydrogen atoms (protium) at carbon twenty (C₂₀) have been replaced with deuterium. The structure of gildeuretinol acetate is shown below:

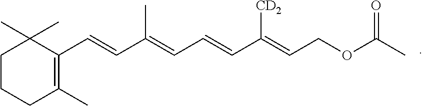

As with retinol acetate (vitamin A acetate), when consumed, the body converts gildeuretinol acetate between its three primary forms (C₂₀D₃-retinyl esters, C₂₀D₃-retinol, and C₂₀D₃-retinaldehyde). C₂₀D₃-retinol is also referred to herein as “gildeuretinol” and C₂₀D₃-retinol acetate as “gildeuretinol acetate”.

Gildeuretinol inhibits the dimerization of vitamin A in the retina, which results in the formation vitamin A dimers. These dimers are believed to contribute to the retinal degeneration which is characteristic of Stargardt disease.

Gildeuretinol takes advantage of deuterium kinetic isotope effects to inhibit the dimerization of vitamin A. The abstraction of a hydrogen atom at carbon number 20 is the rate-limiting step in the dimerization of vitamin A. Deuterium incorporation at this position results in a kinetic isotope effect that increases the activation energy required for the hydrogen abstraction. The increased activation energy decreases the dimerization rate of vitamin A in the retina, resulting in fewer vitamin A dimers. Decreased dimer formation is believed to lead to a mitigation of retinal damage and retinal degeneration.

There is a need for dosage regimens for treating Stargardt patients with gildeuretinol acetate.

### SUMMARY OF THE INVENTION

It has now been found that once daily doses of 7 mg and 14 mg of gildeuretinol acetate result in an average percentage of deuterated plasma vitamin A of 75% and 85% of the total vitamin A (as retinol+gildeuretinol), respectively, after 4 weeks of treatment (see Example 1).

It has also been found that once daily doses of 14 mg and 24 mg of gildeuretinol acetate for about 24 months effectively replaced over 80% vitamin A with gildeuretinol and slowed the progression of retinal degeneration in patients with symptomatic Stargardt disease (see Example 2 and FIG. 2). Additionally, patients taking oral daily doses of 14 mg gildeuretinol acetate showed no disease progression over the duration of the clinical trial (Example 2, FIG. 3-5).

Based on these results, this disclosure provides methods for treating Stargardt Disease through the administration of specific dosage regimens of gildeuretinol acetate.

One embodiment of the invention is a method of treating a patient with Stargardt Disease. The method comprises administering to the patient from 10 mg to 35 mg per day of gildeuretinol acetate.

Another embodiment of the invention is a method of delaying the progression of vision loss in a patient with Stargardt Disease. The method comprises administering to the patient from 10 mg to 35 mg per day of gildeuretinol acetate.

Another embodiment of the invention is gildeuretinol acetate for treating a patient with Stargardt Disease. From 10 mg to 35 mg per day of gildeuretinol acetate are to be administered to the patient.

Another embodiment of the invention is gildeuretinol acetate for delaying the progression of vision loss in a patient with Stargardt Disease. From 10 mg to 35 mg per day of gildeuretinol acetate are to be administered to the patient.

Another embodiment of the invention is the use of gildeuretinol acetate for the manufacture of a medicament for treating a patient with Stargardt Disease. From 10 mg to 35 mg per day of gildeuretinol acetate are to be administered to the patient.

Another embodiment of the invention is the use of gildeuretinol acetate for the manufacture of a medicament for delaying the progression of vision loss in a patient with Stargardt Disease. From 10 mg to 35 mg per day of gildeuretinol acetate are to be administered to the patient.

### BRIEF DESCRIPTION OF THE FIGURES

FIG. 1 is a graph showing the percent gildeuretinol (deuterated vitamin A) versus total plasma vitamin A (as gildeuretinol+retinol) over time in months in patients administered 14 mg/day gildeuretinol acetate for 24 month; in patients administered 24 mg/day gildeuretinol acetate for 12 months followed by 14 mg/day gildeuretinol acetate for 12 months; in patients administered placebo for 12 months followed by 14 mg/day gildeuretinol acetate for 12 months; and in patients administered placebo for 24 months.

FIG. 2 is a graph showing the change in size of atrophic retinal lesions over time in patients treated with gildeuretinol acetate (ALK-001) compared with placebo. Dotted line, untreated arm (natural history+placebo): growth rate of 0.24 mm/year; 15 patients taking placebo and 54 natural history cases. Continuous line, gildeuretinol acetate arm: growth rate of 0.19 mm/year; 34 patients taking gildeuretinol acetate. Error bars are 95% confidence intervals.

FIG. 3 is a graph showing the age-matched visual acuity of three asymptomatic Stargardt disease patients taking gildeuretinol acetate 14 mg daily (bold curves) alongside their untreated Stargardt disease siblings. Each symbol represents a visual acuity measurement for each eye. Curves show average visual acuity for both eyes of each patient. Dotted arrows show comparisons between siblings.

FIG. 4 is a graph showing mean retinal sensitivity (dB) over time in three asymptomatic Stargardt disease patients taking gildeuretinol acetate 14 mg daily. Lines show mean retinal sensitivity of both eyes. Lines are piecewise linear regressions with a knot time of 12 months. Each symbol represents a retinal sensitivity measurement for each eye. Patient 1 retinal sensitivity was measured with a Nidek MP-1 (Nidek, Gamagori, Japan). Patients 2 and 3 retinal sensitivity was measured with the CenterVue Macular Integrity Assessment or MAIA (CenterVue, Padova, Italy). To plot Patient 1 on a comparable scale, 4 dB was added to all measured thresholds of Patient 1.

FIG. 5 is a graph showing retinal photoreceptor loss over time in three asymptomatic Stargardt disease patients taking gildeuretinol acetate 14 mg daily. Lines are piecewise linear regressions with a knot time of 6 months and show the mean area of retinal photoreceptor loss of both eyes. Each symbol represents the area of photoreceptor loss for calculated for one eye. To measure photoreceptor loss, the ellipsoid zone layer was evaluated on b-scans acquired on Heidelberg engineering optical coherence tomography (Heidelberg, Germany).

### DETAILED DESCRIPTION OF THE INVENTION

When the disclosed dosage regimens were administered to individuals 12 years or older clinically diagnosed with Stargardt disease with a well-delineated homogeneous atrophic retinal lesion, the growth of the atrophic retinal lesions are slowed by about 21%. Additionally, asymptomatic Stargardt disease patients administered the disclosed dosage regimen showed no vision loss or disease progression over the duration of the treatment.

Disclosed herein are methods of treating a patient with Stargardt disease or delaying the progression of vision loss in a Stargardt patient by administering to the patient 10 mg to 35 mg per day gildeuretinol acetate. Alternatively, 14-24 mg per day of gildeuretinol acetate are administered to the patient. In another alternative, 12 to 16 mg per day of gildeuretinol acetate are administered to the patient. In another alternative, 22 to 26 mg per day of gildeuretinol acetate are administered to the patient. In another alternative, 14 mg per day of gildeuretinol acetate are administered to the patient. In another alternative, 24 mg per day of gildeuretinol acetate are administered to the patient.

In another alternative, 10 mg to 35 mg of gildeuretinol acetate are administered to the patient once daily. In another alternative, 14-24 mg of gildeuretinol acetate are administered to the patient once daily. In another alternative, 12 to 16 mg of gildeuretinol acetate are administered to the patient once daily. In another alternative, 22 to 26 mg of gildeuretinol acetate are administered to the patient once daily. In another alternative, 14 mg of gildeuretinol acetate are administered to the patient once daily. In another alternative, 24 mg of gildeuretinol acetate are administered to the patient once daily.

Preferably, the gildeuretinol acetate is administered orally according to one of the dosage regimens recited in the previous two paragraphs.

Gildeuretinol acetate is a retinol acetate form of vitamin A, in which the three hydrogen atoms at carbon twenty have been replaced or enriched with deuterium. The structure of gildeuretinol acetate is shown below:

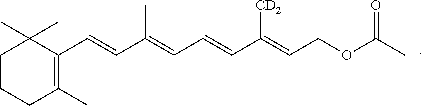

Gildeuretinol acetate is also referred to as “C₂₀-D₃-retinol acetate”. The deuterium enrichment in gildeuretinol acetate is at least 90.0%; at least 95.0%; at least 97.0%; or at least 98.0%. For example, the deuterium enrichment is from 90.0% to 99.8%; 95.0% to 99.8%; 97.0% to 99.8%; 98.0% to 99.8%; 90.0% to 99.5%; 95.0% to 99.5%; 97.0% to 99.5%; or 98.0% to 99.5%. The deuterium enrichment in the gildeuretinol acetate used in Examples 1-3 was from 95.0% to 99.5%. “Deuterium enrichment” is a mole percent and is determined by dividing the number of deuterium atoms at the C₂₀ position by the total number of hydrogen H (as protium)+deuterium atoms at the C₂₀ position. Deuterium enrichment for gildeuretinol acetate can be determined for example by mass spectrometry from the intensities of the 269.2 molecular ion peak (C₂₀H₃ vitamin A), the 270.2 molecular ion peak (C₂₀H₂D vitamin A), the 271.2 molecular ion peak (C₂₀HD₂vitamin A) and the 272.2 (C₂₀D₃ vitamin A) molecular ion peak.

“Treating” a Stargardt disease patient according to a disclosed dosing regimen includes improving, slowing or delaying the progression of symptoms associated with Stargardt disease. “Treating” an asymptomatic Stargardt patient according to a disclosed dosing regimen includes delaying the onset of symptoms associated with Stargardt disease and/or delaying the progression of Stargardt disease patients.

Suitable formulations for gildeuretinol acetate are as described in U.S. RE47,045, the entire teachings of which are incorporated herein by reference. Gildeuretinol acetate can be administered by any suitable means, including orally, subcutaneously, parenterally, intravenously or by injection, topically (including for example as an eyedrop, implant, drug-eluting or other device, intravitreal, suprachoroidal or other injections directly into the eye), but is typically administered orally, as a pill, for example a tablet, a capsule or a softgel. In one aspect, the gildeuretinol acetate is administered orally as a pill. In one aspect, the pill is taken daily. In one aspect, the pill comprises gildeuretinol acetate dissolved in soybean oil and stabilized with an antioxidant. In one example, the antioxidant is butylated hydroxytoluene comprising about 0.1% to 0.2% by weight of the oil. In another aspect, about 10 to 24 mg of gildeuretinol acetate is dissolved into about 400 mg of the oil.

“Stargardt disease” or “Stargardt” as used herein, refers to the diagnosis made by a physician of a patient. Synonyms of Stargardt disease include, without limitation, Stargardt juvenile macular degeneration, Stargardt juvenile macular dystrophy, Stargardt inherited macular degeneration, juvenile macular degeneration, fundus flavimaculatus.

A “Stargardt disease symptom”, or “symptom” as used herein, refers to a characteristic or feature of Stargardt disease that can be perceived or identified by the patient. In one example, a Stargardt disease symptom is some measurable degree of vision loss. The vision loss associated with Stargardt disease is characterized by. Involuntary eye movements are also a symptom of Stargardt disease.

A patient with “asymptomatic Stargardt disease”, as it is used herein, refers to a patient with Stargardt disease who does not yet have any of the symptoms of Stargardt disease.

The term “symptom”, as it is used herein, does not include those characteristics or features of Stargardt disease that can be measured or identified by a physician or other medical professional, but cannot be perceived or identified by loss of central vision, flashes of light, blurred vision, difficulty in seeing in dim lighting or at night, difficulty in distinguishing colors, difficulty in adjusting to the dark, difficulty in adjusting to bright light exposure difficulty tracking moving objects, difficulty in reading, writing, or recognizing faces due to vision loss, and blind spots in the field of vision of the patient. Such characteristics and features of Stargardt disease that are measurable or identifiable only by a physician or other medical professional but not by the patient are referred to herein as “Diagnostic Indicators” of Stargardt disease. Often times, Diagnostic Indicators precede the appearance of symptoms. Therefore, a person who is asymptomatic with respect to Stargardt disease can be diagnosed with Stargardt disease if one or more of these Diagnostic Indicators are present. The presence of these Diagnostic Indicators is in one aspect identified by visually examining the eye with the aid of a slit lamp or similar imaging device. Imaging devices that image the retina, such as fluorescein angiography (FA), fundus autofluorescence (FAF), color fundus photography (CFP), and optical coherence tomography (OCT) can also identify Diagnostic Indicators of Stargardt disease. Examples of Diagnostic Indicators include retinal atrophic lesions, retinal flecks, pigment migration, retinal thinning, ellipsoid zone loss, abnormal or thickened external limiting membrane, retinal atrophy, areas of hypo and hyper-autofluorescence and maculopathy. Often times, Diagnostic Indicators first appear in the parafoveal region.

Retinal atrophic regions, flecks, and other Diagnostic Indicators are also present when Stargardt disease becomes symptomatic and vision loss occurs. The size and/or growth of retinal atrophic regions and flecks can be used as a measure of disease progression.

Stargardt disease is also associated with certain genetic abnormalities known as “mutations” or “variants”. The most common example of a mutation that causes Stargardt disease is when one or more mutations are found in the ATP binding cassette subfamily A member 4 gene (ABCA4), which is a gene that encodes a membrane-associated protein belonging to the superfamily of ATP-binding cassette (ABC) transporters. ABC proteins transport various molecules across extra- and intracellular membranes. ABCA4 mutations that are associated with retinal degeneration and Stargardt disease can be found in databases, including The Genome Aggregation Database (gnomAD), the Leiden Open Variation Database (LOVD), ClinVar, the Exome Aggregation Consortium (ExAC) and The Human Gene Mutation Database (HGMD®). In one aspect, the Stargardt disease patient being treated according to the disclosed dosing regimen has an ABCA4 mutation.

A mutation or variant that is associated with Stargardt disease, such as a mutation in the ABCA4 gene, is referred to herein as a “Stargardt Associated Mutation”. Not all mutations are pathogenic (or disease-causing). A “pathogenic mutation” or “disease-causing mutation”, as used herein, is a mutation determined to be responsible for the Stargardt disease in a patient, or is being used to diagnose Stargardt disease in a patient, or is one allowing a physician to make a treatment decision of a patient. Pathogenicity of novel mutations can be evaluated using a variety of criteria including for example the Standards and Guidelines for the Interpretation of Sequence Variants from the American College of Medical Genetics and Genomics (ACMG) or the Association for Molecular Pathology.

“Biallelic”, as used herein, shall designate a patient who has two or more mutations.

Asymptomatic Stargardt patients can be identified through routine screening of patients for a Diagnostic Indicator or a Stargardt Associated Mutation. Treatment with gildeuretinol acetate, as described herein, can be initiated once a patient is identified as having one or more Diagnostic Indicators or a Stargardt Associated Mutation. Alternatively, treatment with gildeuretinol acetate, as described herein, can be delayed up until the time the patient is identified as having both a Diagnostic Indicator and a Stargardt Associated Mutation. For example, in those instances in which the patient is identified as having a Stargardt Associated Mutation but not a Diagnostic Indicator, treatment with gildeuretinol acetate, as described herein, can be delayed up until the time the presence of a Diagnostic Indicator is detected. Patients with a Stargardt Associated Mutation who do have a Diagnostic Indicator should be routinely monitored for the development of a Diagnostic Indicator and the disclosed gildeuretinol acetate treatment initiated before the onset of Stargardt disease symptoms. As shown in Example 3, the onset of Stargardt disease symptoms can be prevented at least for significant periods of time if treatment is begun when the patient is asymptomatic. Patients who are at risk of Stargardt disease, i.e., patients with a family history of Stargardt disease or a family history of having a Stargardt Associated Mutation, should be screened for Stargardt Associated Mutation and Diagnostic Indicators at an early age before the onset of symptoms.

Genotyping technologies for detecting a Stargardt Associated Mutation include hybridization and/or PCR-based methods, such as iPLEX, pyrosequencing, microarray, and next-generation sequencing. The selection of genotyping techniques depends on parameters such as the amount (i.e., the number of genetic markers) and type (i.e., precision) of genetic information needed, the size of the cohort (i.e., the number of individuals), the computational capacity available (e.g., pre-processing, post-processing), and the financial considerations. Preferably, the genotyping should be able to detect non-coding variants deeper than ±20 base pairs from the exon-intron boundary. These genotyping methods make use of DNA sampled from the potential patient, including blood, saliva, hair, or paraffin-embedded biopsy tissues. Methods for DNA isolation have been described. DNA isolation from the biological samples involves extracting chromosomal DNA from the cellular nuclei and removing proteins and cell debris to yield a DNA sample.

Gildeuretinol acetate can be prepared according to methods described in U.S. RE47,045, and the following references include procedures that can be useful in preparing gildeuretinol: Pardoen et al., Can. J. Chem. 63:1431 (1985); Bonrath et al., Org. Process Res. Dev. 27:1557 (2023); and Bergen et al., Journal of Labelled Compounds XXV:11 (1987).

“Patient” refers to a human patient.

### EXEMPLIFICATION

The following examples are offered to illustrate exemplary embodiments of the invention and do not define or limit its scope.

EXAMPLE 1: Administering daily doses ranging from approximately 7 to 14 mg of gildeuretinol acetate resulted in an average percentage of deuterated plasma vitamin A of about 75% and 85%, respectively, after about 4 weeks of treatment.

### Methods

Study Design: This was an open-label, non-randomized, multiple dose study in healthy volunteers aged 21 to 70 years of age (inclusion criteria). Subjects who met all entrance criteria at screening were invited to receive the study medication. There were six treatment arms: 2 mg/day, 3.5 mg/day, 5 mg/day, 7 mg/day, 10 mg/day and 14 mg/day gildeuretinol acetate. For the four lower dose groups, the dose regimen was one capsule per day, orally, to be taken at home, preferably in the evening unless a different time was preferred by the subject. For the two highest doses, the dose regimen was two capsules per day, one in the morning and one in the evening, although subjects with a preference to take both capsules at once were offered to do so. After taking the 1^{st} dose at the study site, subjects were invited to return two and four weeks after starting the treatment. Each visit had an authorized window of ±1 day.

The clinical staff made periodic calls to monitor adverse events and ensure subject compliance. In addition, a text message was sent daily to remind subjects to take their medication. Subjects were instructed to avoid foods that contain large amounts of vitamin A (such as liver-based products), including supplements containing vitamin A, and to report their vitamin A intake by answering a daily text message diary. Fruits and vegetables were permitted without restriction.

### Pharmacokinetic Evaluations:

Deuterated retinol (gildeuretinol) (“D₃”) and non-deuterated retinol (“Do”) and other vitamin A metabolites were measured to calculate the percentage of deuterated vitamin A in plasma. For all treatment groups, plasma samples were collected at baseline, 14±1 days (“2 weeks”) and 28±1 days (“4 weeks”) after initiation of treatment. Blood samples were usually collected following at least 10 hours of fasting (including last gildeuretinol acetate intake), preferably in the morning. For the 7 mg/day and the 14 mg/day groups only, after the first and last dose, blood samples were collected every 4 hours for 24 hours. A liquid chromatography/mass spectrometry assay was used to measure the metabolite levels.

### Results

Subject Disposition: Forty-five subjects were enrolled. Thirty-seven subjects were assigned to one of the six treatment groups; the remaining eight subjects, enrolled as back-ups, were withdrawn prior to receiving a dose. The 37 treated subjects completed the treatment part of the study. No subject discontinued the treatment. Disposition of the 37 subjects is shown in Table 1.

TABLE 1
Subject Disposition

| | | 2 | 3.5 | 5 | 7 | 10 | 14 |
|---|---|---|---|---|---|---|---|
| Number of subjects | Total | mg/day | mg/day | mg/day | mg/day | mg/day | mg/day |
| Screened | 45 | | | | | | |
| Withdrawn before treatment | 8 | | | | | | |
| Total assigned to treatment | 37 | | | | | | |
| Assigned to treatment group | 37 | 7 | 6 | 6 | 6 | 6 | 6 |
| Treated | 37 | 7 | 6 | 6 | 6 | 6 | 6 |
| Completed the treatment | 37 | 7* | 6 | 6 | 6* | 6* | 6 |
| Still on treatment | 0 | 0 | 0 | 0 | 0 | 0 | 0 |
| Discontinued during treatment | 0 | 0 | 0 | 0 | 0 | 0 | 0 |

Note:
All subjects completed the treatment.

### Pharmacokinetic Results:

Table 2 provides % D₃ Retinol (gildeuretinol, i.e. the percentage of deuterated retinol (gildeuretinol) over total retinol (as deuterated+non-deuterated) in plasma measured 12-24 hours following gildeuretinol acetate intake.

TABLE 2
Percentage of deuterated retinol over total retinol in plasma of fasting subjects

| | % gildeuretinol over retinol + gildeuretinol | | | | | |
|---|---|---|---|---|---|---|
| | 2 | 3.5 | 5 | 7 | 10 | 14 |
| Visit | mg/day | mg/day | mg/day | mg/day | mg/day | mg/day |
| Baseline | 0% | 0% | 0% | 0% | 0% | 0% |
| 24 hours post 1st dose | NM | NM | NM | 27% | NM | 41% |
| After 2 weeks of treatment | 27% | 39% | 53% | 55% | 66% | 64% |
| After 4 weeks of treatment | 31% | 46% | 51% | 63% | 72% | 79% |

NM: not measured

Pharmacokinetic Results (integrated levels over 24 hours post dosing): total vitamin A in plasma, both deuterated (gildeuretinol) and non-deuterated (retinol), was measured every 4 hours following administration of 7 mg or 14 mg at the first dose, and following administration of the 28^{th} dose (4 weeks of daily treatment). The area under the curve (AUC) was calculated and the % deuterated % D₃ of total vitamin A (as retinol and gildeuretinol) was computed as an average over 24 hours. Results are presented below in Table 3.

TABLE 3
Area under the curve (ng/mL · hr) for vitamin A (primarily
retinol) and deuterated vitamin A (primarily gildeuretinol) analytes
following initial (day 1) and final (day 28) 7 mg dose of gildeuretinol
acetate. Percentages of deuterated vitamin A over a 24-hour period
are showed in the last column (% D3 Vitamin A).

| | | | AUC | AUC D3 | |
|---|---|---|---|---|---|
| | | | Vitamin A | Vitamin A | % D3 |
| Group | | Dose | (ng/mL · hr) | (ng/mL · hr) | Vitamin A |
| 7 | mg/day | 1st dose | 9,621 | 6,022 | 38% |
| | | 28th dose | 4,689 | 13,824 | 75% |
| 14 | mg/day | 1st dose | 8,355 | 9,407 | 52% |
| | | 28th dose | 2,976 | 18,093 | 85% |

AUC: Area under the curve

EXAMPLE 2: Administering daily doses of gildeuretinol acetate ranging from approximately 14 to 24 mg of gildeuretinol acetate replaces more than about 80% of the serum vitamin A with gildeuretinol within a matter of weeks. 24 months of daily gildeuretinol acetate at 14 to 24 mg slows retinal degeneration.

### Methods

### Study Design

The Tolerability and Effects of gildeuretinol acetate in Stargardt Disease (Clinical trial group 1 also known as TEASE-1) was a 24-month, multicenter, double-masked, placebo-controlled trial designed to evaluate the efficacy and safety of daily oral gildeuretinol acetate in slowing the growth of atrophic retinal lesions in Stargardt disease patients. The trial was conducted at multiple sites in the United States (NCT02402660).

The study protocol was approved by independent institutional review boards. The trial was conducted in accordance with the principles of the Declaration of Helsinki, International Conference on Harmonization E6 Good Clinical Practice Consolidated Guideline, and other relevant regulations. All participants provided written informed consent prior to screening or initiation of any study-related procedures and received visit-based reimbursement as approved by the independent institutional review boards. No additional stipends or incentives were provided.

### Patients

Patients 12 years or older, clinically diagnosed with Stargardt disease, with at least one ABCA4 mutation, at least one eye presenting with well-demarcated areas of reduced fundus autofluorescence, and with any visual acuity, were eligible.

### Randomization, Masking, and Treatments

Patients were randomized to gildeuretinol acetate 14 mg capsules, gildeuretinol acetate 24 mg capsules, or placebo capsules, to be taken once daily for one year. After one year, 50% of randomly selected patients receiving placebo were crossed over in a 1:1 ratio to gildeuretinol acetate 14 mg capsules or gildeuretinol acetate 24 mg capsules for a second year. If a masked interim review of plasma gildeuretinol levels indicated that the 14 mg and 24 mg doses achieved the same average percentage replacement of plasma vitamin A, patients initially randomized to the 24 mg dose received the 14 mg dose during the second year.

Gildeuretinol acetate and placebo capsules were identical appearing capsules. Patients were advised to avoid vitamin A-containing supplements and liver-based products. Fruits and vegetables contain beta-carotene, not preformed vitamin A, and could be consumed without limitation. Trial visits occurred at 8 weeks and 6, 12, 18, and 24 months following screening and randomization.

### Natural History Cases

Deidentified retinal images of patients not enrolled in the trial were included as a natural history cohort. This natural history cohort comprised patients diagnosed with Stargardt disease of any age, with two or more longitudinal fundus autofluorescence images taken at least four months apart over a maximum period of 2.5 years.

### Primary Efficacy Endpoint

The prespecified primary efficacy endpoint was the growth rate of the square root area of atrophic retinal lesions measured by fundus autofluorescence retinal imaging comparing gildeuretinol versus the untreated arm (placebo combined with natural history). Only well-delineated and homogeneous retinal atrophic lesions≥0.2 mm² with a summed area≥0.5 mm² were included in the primary analysis.

### Statistical Analysis

The primary endpoint analysis used a linear mixed-effect model (LMEM) with repeated measures, conferring a higher power to detect smaller effects. All observed data, including both eyes, were utilized. Treatment duration was computed as a continuous measure for each visit based on years since randomization. Adjustments for baseline retinal atrophy used the baseline atrophic retinal lesion size as a fixed effect in the LMEM. Additional fixed effects included treatment arm, treatment duration, and interaction between the treatment arm and treatment duration. Random effects included patient, eye, grader to adjust for correlation between eyes within a patient, graders' assessment of an eye, and observations of the same eye over time. A random effect slope term was added to the effect of treatment duration. Patients who crossed over to gildeuretinol acetate after year one were included in the untreated arm from baseline to year one, then in the gildeuretinol acetate arm from baseline to year one upon crossing over to gildeuretinol acetate. The full analysis set (FAS) comprised all randomized patients who received at least one dose of the study drug.

Growth rates of atrophic retinal lesions were computed as two segmented lines over the 24-month treatment period; the first segment was between 0 and 6 months, and the second was between 6 and 24 months. All available data between 0 and 24 months was used to compute the slope of each segment. The primary efficacy endpoint was the slope of the second segment. No constraints were imposed on the Y-intercept. All growth rates were calculated on a per-year basis.

No imputations were made for missing data because the LMEM yields valid inferences under the assumption of a missing-at-random mechanism. The two-sided P-value of the primary efficacy endpoint was calculated using Kenward-Roger's method for handling denominator degrees of freedom and small samples. The goodness of fit of the linear mixed-effect model was provided as R-squared (R²). Analyses were conducted in R (R Core Team, 2020). Prespecified, unadjusted sensitivity analyses of the primary efficacy endpoint included growth rates of untransformed areas of retinal atrophy and growth rates using a single-slope from baseline until the last available visit. Post-hoc unadjusted analyses were performed to evaluate the potential contribution of patient characteristics.

### Results

The trial was conducted between August 2015 and December 2020. Fifty-three patients were screened, and 50 were randomized to gildeuretinol acetate 14 mg (n=15), gildeuretinol acetate 24 mg (n=15) or placebo (n=20). After one year, 10 placebo patients switched to gildeuretinol acetate. Four patients dropped out, unrelated to treatment: three from the gildeuretinol acetate 14 mg group and one from the placebo group. All collected data from dropouts were utilized. Seven patients had summed atrophic retinal lesions smaller than <0.5 mm² and thus were excluded from analysis as specified in the statistical analysis plan: three from the gildeuretinol acetate group, three from the crossover group, and one from the placebo group. One patient in the placebo group was excluded because they had no follow-up data.

A total of 63 natural history cases were received from five centers. One case was excluded because the patient was randomized in the trial, one had a PRPH2 mutation, one had histoplasmosis, and one had atrophic lesions outside the imaging field. Four cases were excluded because they lacked follow-up within 4 months, and one because it had a follow-up visit over 2.5 years apart.

The primary efficacy analysis included 96 patients: 42 randomized patients and 54 natural history cases. Overall, demographic and clinical characteristics at baseline were similar in the two groups. The median age was 55, and 56% of patients were female. Of the randomized patients, 41 had two or more ABCA4 mutations, and one had one ABCA4 mutation. Of the natural history cases, 47 had two or more ABCA4 mutations, 3 had one mutation, and genotyping was unavailable for 7. Three to seven time points for at least one eye were available for 39 cases, while 16 cases had two time points for at least one eye.

### Plasma Gildeuretinol

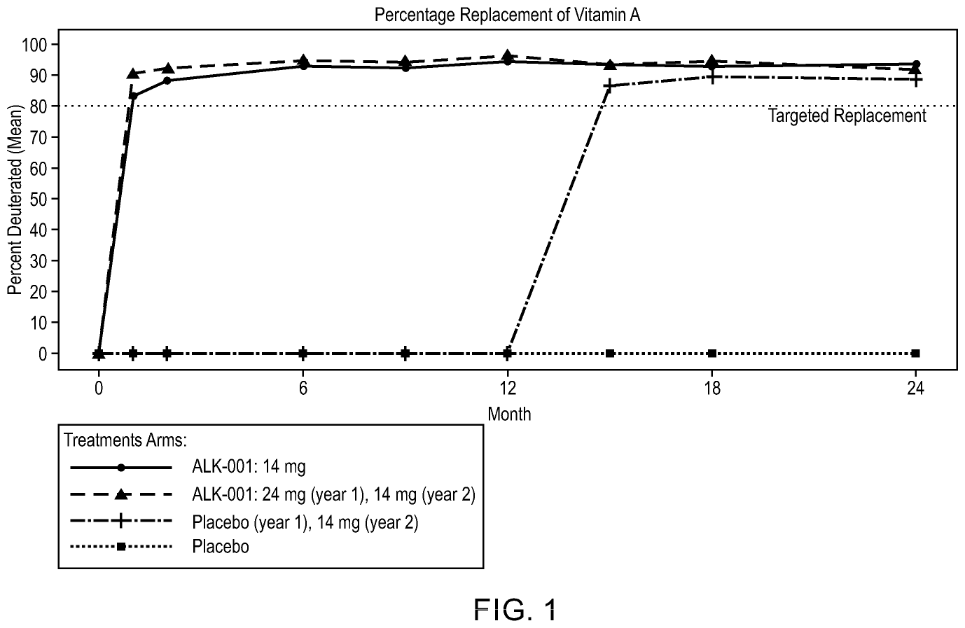

In the 8-week pharmacokinetic analysis, the percentage of gildeuretinol over total vitamin A (as retinol+gildeuretinol) in the 14 mg and 24 mg cohorts was equivalent (87% and 92%, respectively). Thus, all treated patients received gildeuretinol acetate 14 mg for the second year of treatment, and patients taking any dose formed the gildeuretinol acetate arm. A greater than 80% replacement was sustained throughout the treatment period. After six months, over 80% of plasma vitamin A was replaced in 39 out of 40 patients receiving gildeuretinol acetate (FIG. 1). One crossover patient did not achieve 80% replacement due to non-compliance. Total plasma vitamin A and retinoic acid, the combined vitamin A and gildeuretinol-derived forms, were the same between gildeuretinol acetate and placebo groups. No patients receiving placebo had detectable levels of gildeuretinol.

### Primary Endpoint

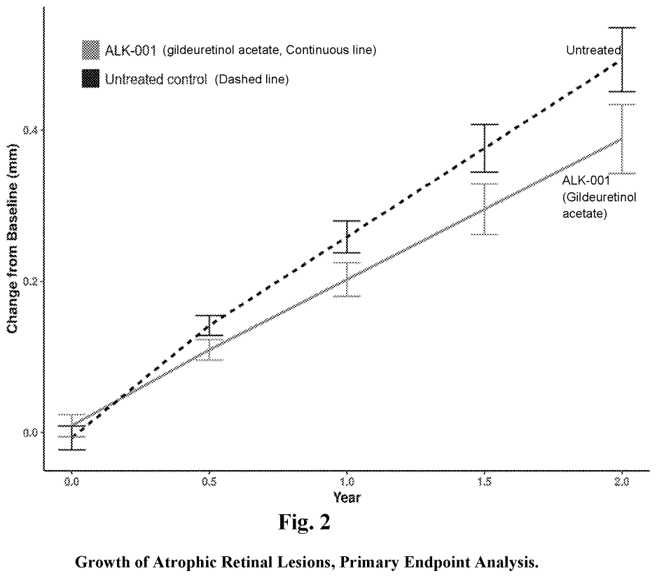

The growth rate of atrophic retinal lesions was 0.19 mm/year with gildeuretinol acetate and 0.24 mm/year with control, a difference of 0.05 mm/year (95% confidence interval [CI], 0.03 to 0.07), corresponding to a relative 21% slower growth rate in patients treated with gildeuretinol acetate (P<0.001, R²=0.9996) (FIG. 2).

The significant positive treatment effect persisted across different calculations for estimating the growth rate of atrophic retinal lesions and remained consistent when considering various scenarios, such as excluding the entire natural history cohort, including or excluding natural history cases with two-time points, cases with a single identified ABCA4 mutation, or cases with no genetic information (Table 4).

TABLE 4
Estimated Treatment Effects Using Different Calculations
and Populations Estimation Methods and/Population.

| | Percentage |
|---|---|
| | Slowing of |
| | Atrophic |
| | Retinal |
| | Lesion |
| | Growth Rate |
| | With |
| | Gildeuretinol |
| Estimation Method/Population | Acetate |
| Using untransformed areas of atrophic lesions (mm²) | 28% |
| Using a single-slope from 0 to 24 months (mm²) | 31% |
| Crossover patients: 12 months placebo vs. 12 | 21% |
| months treatment | |
| Excluding 13 natural history cases without any | 20% |
| confirmed ABCA4 mutations and 6 natural histories | |
| with one confirmed mutation | |
| Excluding 2 natural history cases < 18 years old, 9 | 21% |
| natural histories > 60 years old | |
| Excluding 16 natural histories with only two follow | 20% |
| up time points | |
| Excluding natural history, randomized patients, only, | 23% |
| two slopes (mm²) | |
| Excluding natural history, randomized patients only, | 30% |
| one slope (mm²) | |
| Excluding 3 randomized patients with one | 20% |
| confirmed mutation | |

Gildeuretinol acetate met its prespecified primary efficacy endpoint, slowing the mean growth rate of atrophic retinal lesions relative to untreated patients composed of a placebo and natural history.

### Safety

No serious adverse events were associated with gildeuretinol acetate and no patient discontinued treatment due to side effects. All adverse events were mild or moderate, and no association was observed between treatment groups and the frequency of adverse events (Table 5). No changes in visual acuity were observed. By physical examination, electrocardiogram, vital signs, hematology, biochemistry and lipid panels, no changes were attributed to gildeuretinol acetate. Blurred optic disc margins were observed in one patient receiving the 24 mg dose upon ocular exam at the 12-month visit, associated with complaints of transient visual obscuration. Elevated intracranial pressure was measured, and papilledema Grade 1 (modified Frisen scale) was diagnosed. This event resolved upon temporary interruption of gildeuretinol acetate and treatment with acetazolamide. The patient resumed treatment and completed the trial without recurrence.

TABLE 5
All adverse events observed in the TEASE-1 trial, presented
by MedDRA System Organ Class and Preferred Term. Coded using
MedDRA version 22.0. Interim data as of December 2020.

| | | Number of patients experiencing event (%) | | | |
|---|---|---|---|---|---|
| | | gildeuretinol | gildeuretinol | | |
| | | acetate | acetate | | |
| System Organ | | 14 mg | 24 mg | Crossover | Placebo |
| Class | Preferred Term | (N = 15) | (N = 15) | (N = 10) | (N = 10) |
| Overall | Overall | 12 (80) | 13 (86.7) | 8 (80) | 9 (90.0) |
| Ear and labyrinth | Ear discomfort | 0 | 1 (6.7) | 0 | 0 |
| disorders | Ear pain | 0 | 0 | 0 | 1 (10.0) |
| Endocrine | Hypothyroidism | 0 | 1 (6.7) | 0 | 0 |
| disorders | | | | | |
| Eye disorders | Blepharospasm | 0 | 0 | 1 (10.0) | 0 |
| | Diplopia | 0 | 1 (6.7) | 0 | 0 |
| | Dry eye | 0 | 1 (6.7) | 0 | 1 (10.0) |
| | Eye irritation | 1 (6.7) | 0 | 0 | 0 |
| | Eye pain | 0 | 1 (6.7) | 0 | 1 (10.0) |
| | Eye pruritus | 0 | 1 (6.7) | 0 | 0 |
| | Photophobia | 1 (6.7) | 0 | 0 | 1 (10.0) |
| | Photopsia | 1 (6.7) | 0 | 0 | 0 |
| | Punctate keratitis | 1 (6.7) | 0 | 0 | 0 |
| | Vision blurred | 1 (6.7) | 0 | 1 (10.0) | 0 |
| | Visual impairment | 1 (6.7) | 0 | 0 | 1 (10.0) |
| | Vitreous floaters | 1 (6.7) | 0 | 1 (10.0) | 0 |
| Gastrointestinal | Abdominal | 0 | 1 (6.7) | 1 (10.0) | 0 |
| disorders | discomfort | | | | |
| | Abdominal | 0 | 1 (6.7) | 0 | 0 |
| | distension | | | | |
| | Abdominal pain | 0 | 1 (6.7) | 0 | 0 |
| | upper | | | | |
| | Bowel movement | 0 | 1 (6.7) | 0 | 0 |
| | irregularity | | | | |
| | Constipation | 0 | 1 (6.7) | 0 | 0 |
| | Diarrhoea | 1 (6.7) | 0 | 1 (10.0) | 0 |
| | Dry mouth | 1 (6.7) | 0 | 0 | 0 |
| | Faeces soft | 1 (6.7) | 0 | 0 | 0 |
| | Lip swelling | 0 | 0 | 0 | 1 (10.0) |
| | Nausea | 0 | 1 (6.7) | 0 | 0 |
| | Tongue dry | 1 (6.7) | 0 | 0 | 0 |
| | Toothache | 0 | 0 | 0 | 1 (10.0) |
| General disorders | Death | 1 (6.7) | 0 | 0 | 0 |
| and administration | Inflammation | 0 | 0 | 0 | 1 (10.0) |
| site conditions | Pain | 2 (13.3) | 0 | 0 | 0 |
| | Pyrexia | 0 | 1 (6.7) | 0 | 0 |
| Infections and | Bronchitis | 0 | 0 | 0 | 1 (10.0) |
| infestations | Conjunctivitis | 1 (6.7) | 0 | 0 | 0 |
| | Ear infection | 0 | 0 | 1 (10.0) | 0 |
| | Gastroenteritis | 1 (6.7) | 0 | 1 (10.0) | 0 |
| | Gastroenteritis viral | 0 | 1 (6.7) | 0 | 0 |
| | Herpes zoster | 1 (6.7) | 0 | 0 | 0 |
| | Hordeolum | 0 | 1 (6.7) | 0 | “0) |
| | Influenza | 0 | 1 (6.7) | 0 | 0 |
| | Laryngitis | 1 (6.7) | 0 | 0 | 0 |
| | Nasopharyngitis | 2 (13.3) | 3 (20.0) | 1 (10.0) | 5 (50.0) |
| | Pharyngitis | 1 (6.7) | 0 | 0 | 0 |
| | streptococcal | | | | |
| | Pneumonia | 0 | 0 | 1 (10.0) | 0 |
| Infections and | Sialoadenitis | 1 (6.7) | 0 | 0 | 0 |
| infestations (cont.) | Sinusitis | 1 (6.7) | 0 | 0 | 0 |
| | Skin infection | 0 | 0 | 0 | 1 (10.0) |
| | Tooth abscess | 0 | 1 (6.7) | 0 | 0 |
| | Upper respiratory | 1 (6.7) | 1 (6.7) | 2 (20.0) | 2 (20.0) |
| | tract infection | | | | |
| | Vulvovaginal | 0 | 1 (6.7) | 0 | 0 |
| | candidiasis | | | | |
| Injury, poisoning | Ankle fracture | 1 (6.7) | 0 | 0 | 0 |
| and procedural | Back injury | 0 | 0 | 0 | 1 (10.0) |
| complications | Cartilage injury | 1 (6.7) | 0 | 0 | 0 |
| | Conjunctival | 0 | 1 (6.7) | 0 | 0 |
| | laceration | | | | |
| | Face injury | 1 (6.7) | 0 | 0 | 0 |
| | Fall | 1 (6.7) | 1 (6.7) | 0 | 0 |
| | Hand fracture | 1 (6.7) | 0 | 0 | 0 |
| | Hypobarism | 0 | 1 (6.7) | 0 | 0 |
| | Muscle strain | 0 | 0 | 0 | 1 (10.0) |
| | Radius fracture | 1 (6.7) | 0 | 0 | 0 |
| | Subdural haematoma | 1 (6.7) | 0 | 0 | 0 |
| | Wrist fracture | 0 | 0 | 0 | 1 (10.0) |
| Investigations | Alanine | 1 (6.7) | 0 | 1 (10.0) | 0 |
| | aminotransferase | | | | |
| | increased | | | | |
| | Aspartate | 1 (6.7) | 0 | 1 (10.0) | 0 |
| | aminotransferase | | | | |
| | increased | | | | |
| | Blood cholesterol | 0 | 1 (6.7) | 0 | 0 |
| | increased | | | | |
| | Blood creatinine | 1 (6.7) | 0 | 0 | 0 |
| | increased | | | | |
| | Blood iron | 0 | 1 (6.7) | 0 | 0 |
| | decreased | | | | |
| | Blood pressure | 0 | 1 (6.7) | 0 | 0 |
| | increased | | | | |
| | Blood triglycerides | 1 (6.7) | 0 | 0 | 0 |
| | increased | | | | |
| | Electrocardiogram | 0 | 0 | 1 (10.0) | 0 |
| | abnormal | | | | |
| | Low density | 1 (6.7) | 0 | 0 | 0 |
| | lipoprotein increased | | | | |
| | Vitamin D decreased | 2 (13.3) | 1 (6.7) | 0 | 0 |
| Metabolism and | Vitamin B12 | 1 (6.7) | 0 | 0 | 0 |
| nutrition disorders | deficiency | | | | |
| Musculoskeletal | Arthralgia | 1 (6.7) | 1 (6.7) | 1 (10.0) | 1 (10.0) |
| and connective | Arthritis | 0 | 0 | 1 (10.0) | 0 |
| tissue disorders | Back pain | 0 | 1 (6.7) | 0 | 1 (10.0) |
| | Muscle spasms | 1 (6.7) | 0 | 0 | 1 (10.0) |
| | Musculoskeletal | 1 (6.7) | 1 (6.7) | 0 | 1 (10.0) |
| | pain | | | | |
| | Neck pain | 0 | 0 | 0 | 1 (10.0) |
| | Osteoporosis | 0 | 1 (6.7) | 0 | 0 |
| | Pain in extremity | 1 (6.7) | 2 (13.3) | 0 | 0 |
| | Pain in jaw | 0 | 0 | 0 | 1 (10.0) |
| | Plantar fasciitis | 1 (6.7) | 0 | 0 | 0 |
| | Rotator cuff | 0 | 1 (6.7) | 0 | 0 |
| | syndrome | | | | |
| | Sjogren's syndrome | 1 (6.7) | 0 | 0 | 0 |
| | Tendonitis | 1 (6.7) | 0 | 0 | 0 |
| Nervous system | Headache | 1 (6.7) | 2 (13.3) | 0 | 1 (10.0) |
| disorders | Intracranial pressure | 0 | 1 (6.7) | 0 | 0 |
| | increased | | | | |
| | Nerve compression | 0 | 0 | 0 | 1 (10.0) |
| | Neuralgia | 1 (6.7) | 0 | 0 | 0 |
| | Syncope | 0 | 1 (6.7) | 0 | 0 |
| | Thoracic outlet | 0 | 0 | 0 | 1 (10.0) |
| | syndrome | | | | |
| | Visual field defect | 0 | 0 | 1 (10.0) | 0 |
| Psychiatric | Abnormal dreams | 0 | 1 (6.7) | 0 | 0 |
| disorders | | | | | |
| Psychiatric | Depression | 1 (6.7) | 0 | 0 | 0 |
| disorders (cont.) | Insomnia | 1 (6.7) | 0 | 0 | 2 (20.0) |
| | Irritability | 0 | 0 | 0 | 1 (10.0) |
| | Obsessive- | 0 | 0 | 0 | 1 (10.0) |
| | compulsive disorder | | | | |
| Renal and urinary | Nephrolithiasis | 1 (6.7) | 0 | 1 (10.0) | 0 |
| disorders | | | | | |
| Reproductive | Premenstrual | 0 | 1 (6.7) | 0 | 0 |
| system and breast | syndrome | | | | |
| disorders | | | | | |
| Respiratory, | Cough | 0 | 2 (13.3) | 0 | 0 |
| thoracic and | Oropharyngeal pain | 0 | 3 (20.0) | 1 (10.0) | 0 |
| mediastinal | Pleurisy | 0 | 0 | 1 (10.0) | 0 |
| disorders | Sinus congestion | 0 | 1 (6.7) | 0 | 0 |
| Skin and | Alopecia | 2 (13.3) | 0 | 0 | 1 (10.0) |
| subcutaneous | Dry skin | 0 | 1 (6.7) | 0 | 0 |
| tissue disorders | Eczema | 0 | 0 | 1 (10.0) | 0 |
| | Erythema | 0 | 0 | 0 | 1 (10.0) |
| | Hair texture | 0 | 0 | 0 | 1 (10.0) |
| | abnormal | | | | |
| | Rash | 1 (6.7) | 0 | 1 (10.0) | 0 |
| | Xanthelasma | 0 | 1 (6.7) | 0 | 0 |
| Surgical and | Hysterectomy | 0 | 1 (6.7) | 0 | 0 |
| medical | Jaw operation | 0 | 1 (6.7) | 0 | 0 |
| procedures | Knee arthroplasty | 1 (6.7) | 0 | 0 | 0 |
| | Tooth extraction | 0 | 1 (6.7) | 0 | 0 |
| Vascular disorders | Hypertension | 0 | 1 (6.7) | 0 | 2 (20.0) |
| | Peripheral coldness | 0 | 0 | 0 | 1 (10.0) |

### Discussion

In this TEASE-1 study, gildeuretinol acetate met its prespecified primary efficacy endpoint, slowing the mean growth rate of atrophic retinal lesions relative to placebo and natural history. Including the natural history cases expanded the study population, providing a more accurate, truer estimate of the treatment effect. The growth rates of atrophic retinal lesions in the placebo and natural history cases were indistinguishable.

The estimated treatment effect ranged from approximately 20% to 30%, depending on the calculation method. The positive treatment effect when comparing the cross-over patients only (6) months of placebo vs. 12 months of treatment for the same patients) underscores gildeuretinol acetate's capacity to alter the clinical trajectory of Stargardt disease.

In conclusion, oral gildeuretinol acetate, taken once daily for up to 24 months, slowed the expansion of retinal atrophic lesions in individuals with Stargardt disease.

EXAMPLE 3: In individuals with asymptomatic Stargardt Disease, daily oral gildeuretinol acetate 14 mg capsule arrested loss of vision and retinal pathology.

### Methods

### Trial Design and Oversight

The tolerability and effects of gildeuretinol acetate in Stargardt Disease (Clinical trial group 3 also known as TEASE-3) study was an open-label, multicenter, case control study designed to evaluate the efficacy and safety of gildeuretinol acetate on the progression of Stargardt disease in asymptomatic individuals. The trial was conducted at multiple sites in the United States.

To assess the safety and efficacy of gildeuretinol acetate in preventing vision loss in asymptomatic individuals, gildeuretinol acetate 14 mg capsules was administered to individuals showing early signs of retinal damage while maintaining approximately 20/20 vision and no subjective loss of vision. Participants had a family history of Stargardt disease, particularly a two-year older sibling carrying identical genetic mutations and already exhibiting symptoms. Based on the above family history and clinical observations, the individuals were likely to develop retinal lesions, loss of photoreceptors, drops in retinal sensitivity, loss of visual acuity, indicating the onset of symptoms, i.e. a progression to the symptomatic phase, including vision loss within two years, mirroring the progression observed in their older siblings.

Testing individuals for Stargardt disease before they exhibit symptoms (presymptomatic testing) can result in unwarranted stigmatization and emotional stress. Testing asymptomatic individuals in the absence of a potential intervention is, therefore, controversial. All asymptomatic individuals in the study were thus administered gildeuretinol acetate. Specific dose levels were not disclosed to the patients. Disease progression was compared to the symptomatic siblings with identical genetic mutations when aged-matched who had not received gildeuretinol acetate.

The study protocol was approved at each site by independent institutional review boards. The trial was conducted in accordance with the principles of the Declaration of Helsinki, International Conference on Harmonization E6 Good Clinical Practice Consolidated Guideline, and other relevant regulations. All participants provided written informed consent prior to screening or initiation of any study-related procedures and received visit-based reimbursement as approved by the independent ethics committees and institutional review boards. No additional stipends or incentives were provided.

### Patients

Patients 10 years of age and older, clinically diagnosed with Stargardt disease by a physician, diagnosis further supported by a genetic report indicating at least one ABCA4 mutation, exhibiting early signs of maculopathy, but with approximately 20/20 visual acuity and no complaints of visual impairment.

### Treatments

Patients received gildeuretinol acetate 14 mg capsules, to be taken orally once daily. Trial visits occurred every 6 months approximately following screening and treatment start visits.

### Study End Points and Assessments

Disease progression was monitored by best-corrected visual acuity (BCVA) reported in number of Early Treatment Diabetic Retinopathy Study (ETDRS) letters, fundus autofluorescence (FAF) imaging, loss of photoreceptors (as measured by the ellipsoid zone band loss) by optical coherence tomography (OCT) according to literature (1-3) and retinal sensitivity by microperimetry.

### Safety

Ocular safety was assessed by retinal imaging and ocular exam, which included evaluation of the anterior and posterior segments and the lens. Systemic safety was assessed by physical exam, comprehensive clinical labs (biochemistry, hematology, lipids), and 12-lead ECG.

### Statistical Analyses

Measurements from the left and right eye for each patient were averaged for each visit to produce a single statistical unit for both eyes(4). For segmented linear regressions, knot times were selected so that three visits were included in the first segment.

### Results

### Participants

Three asymptomatic patients with Stargardt disease, with approximately 20/20 visual acuity, 13, 17, and 18 years of age, were enrolled. All three patients had one or multiple signs of maculopathy, including thinned outer nuclear layers, granular ellipsoid zone layers, and parafoveal photoreceptor loss by OCT, and hyper-autofluorescent patterns surrounding the foveal center by FAF imaging. Each enrolled patient had a symptomatic Stargardt disease sibling who was about two years older. The older untreated siblings had mild to moderate visual acuity loss, outer retinal atrophy in the foveal region, and a diffuse, mottled autofluorescent pattern with hyper-autofluorescent flecks. Genetic testing further supported that each sibling pair shared the same disease-causing mutations on the ABCA4 gene.

Between April 2017 and December 2019, the three patients took gildeuretinol acetate 14 mg oral capsules daily for two years. After the end of this initial two years of treatment, two patients continued treatment in an extension study and one participant discontinued treatment due to relocation. After the initial two years of treatment, each patient was the same age as when their older sibling started to lose vision. After the additional two years of treatment, corresponding to a total treatment duration of four years with gildeuretinol acetate, the two treated patients were two years older than the age of onset of their symptomatic Stargardt disease sibling.

### Plasma Gildeuretinol

After the first two months of treatment, all three participants achieved an 80% replacement of plasma vitamin A (as retinol+gildeuretinol) with gildeuretinol. Over 80% replacement was sustained throughout the treatment period.

### Best Corrected Visual Acuity

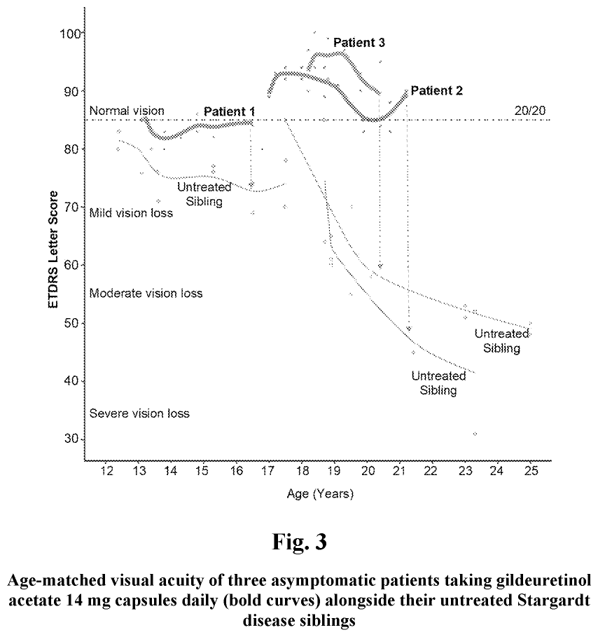

BCVA for the treated siblings remined stable at 88±4 ETDRS letters (~20/20), despite their untreated siblings having mild to moderate, uncorrectable BCVA of 66±10 ETDRS letters (between 20/40 and 20/100), corresponding to an average improvement of 22±13 letters or four lines of vision on the ETDRS eyechart (FIG. 3).

### Fundus Autofluorescence (FAF) Imaging

After two to four years of treatment, none of the three treated asymptomatic Stargardt disease patients exhibited changes by FAF: no retinal flecks appeared, no new areas of increased or decreased autofluorescence formed, and no new atrophy developed. In contrast, the three untreated age-matched siblings all showed year-over-year, progressive worsening by FAF imaging, including expansion of hyperautofluorescent rings, mottled areas, and development of atrophic retinal lesions.

### Photoreceptors Density by Optical Coherence Tomography

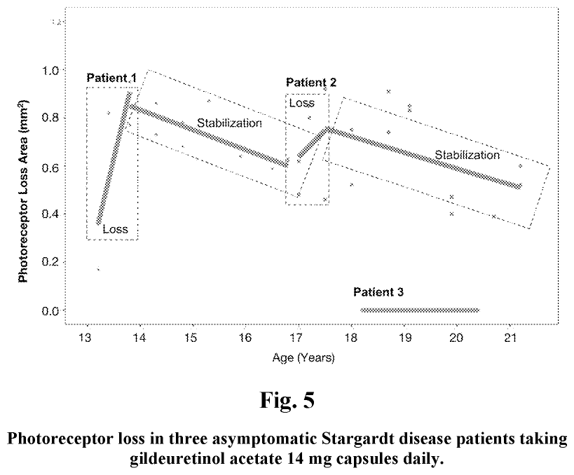

During the first six months of treatment, photoreceptor loss progressed, on average, by approximately 0.18 mm²/year for two treated patents, which was consistent with the natural history and the fact that the disease process had already started at treatment initiation. For the following years of treatment, photoreceptor loss trended toward an improvement (FIG. 5). For the third patient, no photoreceptors were lost during the full treatment period. The average area of photoreceptor loss was limited to 0.36±0.32 mm² or 20% of the foveal area, consistent with the maintenance of visual acuity.

### Retinal Sensitivity by Fundus-Tracking Microperimetry

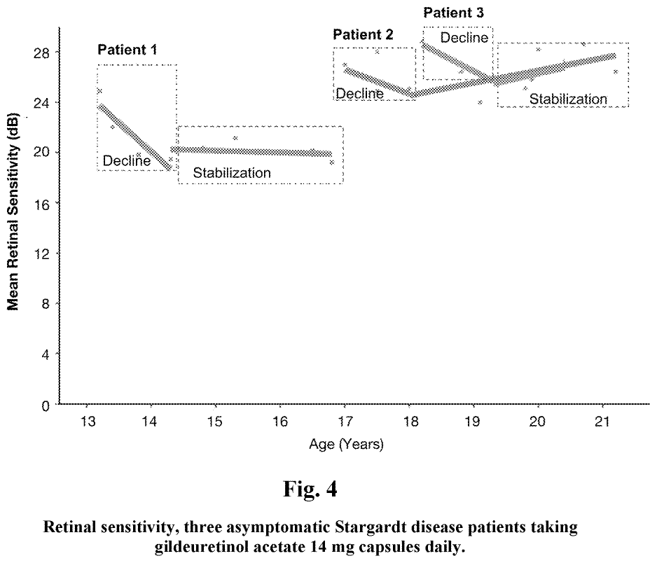

During the first year of treatment, mean retinal sensitivity decreased by an average of −3.1±1.5 dB/year. For the following years of treatment, mean retinal sensitivity trended toward an improvement with an average rate of retinal sensitivity improvement of +0.93±0.7 dB/year (FIG. 4).

### Safety

None of the three treated Stargardt disease asymptomatic patients exhibited adverse reactions linked to either excessive or insufficient vitamin A, such as delays in dark adaptation or night blindness.

### Discussion

In the TEASE-3 study, three asymptomatic Stargardt disease patients with a high likelihood of developing vision loss and symptoms of Stargardt disease, in the next two years, based on their family history and clinical findings, took gildeuretinol acetate 14 mg oral capsules daily for 2 to 4 years. There was no observable disease progression during the treatment period. With 80% replacement of retinol (vitamin A) with gildeuretinol (deuterated vitamin A), the aberrant dimerization of vitamin A that leads to the formation of toxic vitamin A dimers and Stargardt disease is corrected to that of unaffected individuals.

In Stargardt disease, progressive FAF changes occur year-over-year. The absence of FAF changes in all three Stargardt disease patients treated with gildeuretinol acetate contrasts with the natural history of the disease and suggests a treatment-induced arrest, as supported by the stabilization of BCVA. The similar degree of retinal pathology by FAF in historical age-matched siblings with Stargardt disease when both are untreated, further emphasizes the significance of the complete absence of changes by FAF imaging.

Reported rates of photoreceptors lost (Ellipsoid Zone band loss) in individuals with Stargardt disease ranged from −0.33±0.38 to −1.20±1.29 mm²/year. (7-9) Simultaneously, retinal sensitivity is reported to decline at a rate of −2.2±3.0 dB/year. (5) During the first six months to a year of treatment, we observed similar declines in photoreceptor and retinal sensitivity. However, with prolonged treatment both outcomes trended toward an improvement. Evidence from TEASE-1 suggests that administering gildeuretinol acetate later in later disease stages slows disease progression; evidence from TEASE-3 suggests that early administration may arrest disease development.

For individuals with asymptomatic Stargardt Disease, once-daily oral gildeuretinol acetate, 14 mg capsules, taken for up to four years, effectively prevented vision loss and further retinal damage, in effect arresting disease progression for the duration of the intervention.

### REFERENCES

1. Cai C X, Light J G, Handa J T. Quantifying the Rate of Ellipsoid Zone Loss in Stargardt Disease. Am J Ophthalmol. 2018; 186:1-9. Epub 2017/11/12. doi: 10.1016/j.ajo.2017.10.032. PubMed PMID: 29126757.
2. Tanna P, Georgiou M, Strauss R W, Ali N, Kumaran N, Kalitzeos A, Fujinami K, Michaelides M. Cross-Sectional and Longitudinal Assessment of the Ellipsoid Zone in Childhood-Onset Stargardt Disease. Transl Vis Sci Technol. 2019; 8(2):1. Epub 2019/03/06. doi: 10.1167/tvst.8.2.1. PubMed PMID: 30834176; PMCID: PMC6397016.
3. Greenstein V C, Castillejos D S, Tsang S H, Lee W, Sparrow J R, Allikmets R, Birch D G, Hood D C. Monitoring Lesion Area Progression in Stargardt Disease: A Comparison of En Face Optical Coherence Tomography and Fundus Autofluorescence. Transl Vis Sci Technol. 2023; 12(5):2. Epub 2023/05/01. doi: 10.1167/tvst.12.5.2. PubMed PMID: 37126335; PMCID: PMC10153573 Therapeutics, Inc. (F), Emendo (F); W. Lee, None; J. R. Sparrow, None; R. Allikmets, SpliceBio (C), Belite Bio (C), Intergalactic Therapeutics (C), Rectify Pharmaceuticals (C), Shape Therapeutics (C); D. G. Birch, Nacuity Pharmaceuticals (C), Editas (C), ONL (C), AGTC (C, F), Novartis (C), Biogen (F), Ocugen (F), PYC (F), DTx (F); D.C. Hood, Heidelberg Engineering (R, F), Topcon, Inc. (R, F).
4. Karakosta A, Vassilaki M, Plainis S, Elfadl N H, Tsilimbaris M, Moschandreas J. Choice of analytic approach for eye-specific outcomes: one eye or two? Am J Ophthalmol. 2012; 153(3):571-9 el. Epub 2011/11/15. doi: 10.1016/j.ajo.2011.08.032. PubMed PMID: 22078901.
5. Schonbach E M, Strauss R W, Ibrahim M A, Janes J L, Birch D G, Cideciyan A V, Sunness J S, Munoz B, Ip M S, Sadda S R, Scholl H P N, ProgStar Study G. Faster Sensitivity Loss around Dense Scotomas than for Overall Macular Sensitivity in Stargardt Disease: ProgStar Report No. 14. Am J Ophthalmol. 2020; 216:219-25. Epub 2020/03/31. doi: 10.1016/j.ajo.2020.03.020. PubMed PMID: 32222369.

1-16. (canceled)

17. A method of treating a patient with Stargardt disease, comprising administering to the patient from 14 mg to 24 mg per day of gildeuretinol acetate.

18. The method of claim 17, wherein the patient is experiencing partial vision loss from the Stargardt disease.

19. The method of claim 17, wherein the patient is experiencing symptoms of Stargardt disease.

20. The method of claim 17, wherein the patient is not yet experiencing vision loss from the Stargardt disease.

21. The method of claim 17, wherein the patient is asymptomatic for Stargardt disease.

22. The method of claim 17, wherein the patient has a Stargardt Associated Mutation.

23. The method of claim 17, wherein the patient has biallelic Stargardt Associated Mutations.

24. The method of claim 17, wherein the patient has an ABCA4 mutation.

25. The method of claim 17, wherein the patient is experiencing loss of central vision, flashes of light, blurred vision, difficulty in seeing in dim lighting or at night, difficulty in distinguishing colors, difficultly in adjusting to the dark, difficulty in adjusting to bright light exposure difficulty tracking moving objects, difficulty in reading, writing, or recognizing faces due to vision loss, blind spots in the field of vision and/or involuntary eye movements.

26. The method of claim 17, wherein the patient is not yet experiencing loss of central vision, flashes of light, blurred vision, difficulty in seeing in dim lighting or at night, difficulty in distinguishing colors, difficulty in adjusting to the dark, difficulty in adjusting to bright light exposure difficulty tracking moving objects, difficulty in reading, writing, or recognizing faces due to vision loss, blind spots in the field of vision and/or involuntary eye movements.

27. The method of claim 17, wherein the patient is administered 14 mg per day of gildeuretinol acetate.

28. The method of claim 17, wherein the patient is administered 24 mg per day of gildeuretinol acetate.

29. The method of claim 27, wherein gildeuretinol acetate is administered once daily.

30. The method of claim 29, wherein gildeuretinol acetate is administered orally.

31. A method of delaying the progression of vision loss in a patient with Stargardt disease, comprising administering to the patient from 14 mg to 24 mg per day of gildeuretinol acetate.

32. The method of claim 31, wherein the patient is experiencing partial vision loss from the Stargardt disease.

33. The method of claim 31, wherein the patient is experiencing symptoms of Stargardt disease.

34. The method of claim 31, wherein the patient is not yet experiencing vision loss from the Stargardt disease.

35. The method of claim 31, wherein the patient is asymptomatic for Stargardt disease.

36. The method of claim 31, wherein the patient has a Stargardt Associated Mutation.

37. The method of claim 31, wherein the patient has biallelic Stargardt Associated Mutations.

38. The method of claim 31, wherein the patient has an ABCA4 mutation.

39. The method of claim 31, wherein the patient is experiencing loss of central vision, flashes of light, blurred vision, difficulty in seeing in dim lighting or at night, difficulty in distinguishing colors, difficultly in adjusting to the dark, difficulty in adjusting to bright light exposure difficulty tracking moving objects, difficulty in reading, writing, or recognizing faces due to vision loss, blind spots in the field of vision and/or involuntary eye movements.

40. The method of claim 31, wherein the patient is not yet experiencing loss of central vision, flashes of light, blurred vision, difficulty in seeing in dim lighting or at night, difficulty in distinguishing colors, difficulty in adjusting to the dark, difficulty in adjusting to bright light exposure difficulty tracking moving objects, difficulty in reading, writing, or recognizing faces due to vision loss, blind spots in the field of vision and/or involuntary eye movements.

41. The method of claim 31, wherein the patient is administered 14 mg per day of gildeuretinol acetate.

42. The method of claim 31, wherein the patient is administered 24 mg per day of gildeuretinol acetate.

43. The method of claim 41, wherein gildeuretinol acetate is administered once daily.

44. The method of claim 43, wherein gildeuretinol acetate is administered orally.
